(19) United States
(12) Patent Application Publication
(10) Pub. No.: US 2005/0075527 A1
(43) Pub. Date: Apr. 7, 2005

(54) Method and processing equipment for hydrocarbons and for separation of the phases produced by said processing

(75) Inventors: Mathieu Pinault, Lyon (FR); Frederic Hoffmann, Sainte Foy les Lyon (FR); Stephane Kressmann, Serezin Du Rhone (FR); Pierre Porot, Paris (FR); John E. Duddy, Bensalem (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: Institut Francais du Petrole, Rueil Malmaison Cedex (FR)

(21) Appl. No.: 10/373,197

(22) Filed: Feb. 26, 2003

(51) Int. Cl. C07C 7/00; B01J 10/00

(52) U.S. Cl. 585/809; 422/236; 422/188; 422/234

(57) ABSTRACT

Method for processing hydrocarbons comprising at least two processing chambers and means for separating the liquid and gaseous fractions contained in the polyphasic mixture produced in the first processing chamber, said mixture coming from a zone (700) where a batch of liquid hydrocarbons, a gaseous batch and solid particles come into contact, said method comprising separation of liquid and gaseous phases located in the chamber. Equipment comprising: a) At least a first chamber comprising means inside said chamber for separating gaseous and liquid fractions coming from said zone, b) means for conveying said liquid fraction toward a second chamber, c) a second chamber comprising a second contact zone and means of introducing, into said zone, a gaseous batch and a liquid batch comprising at least a portion of the liquid fraction coming from the first chamber.

This invention relates to equipment and a method for processing hydrocarbons comprising at least two chambers or a succession of chambers arranged in series, at least one reaction being produced the most often inside said chambers, incorporating at least one solid phase, at least one liquid phase and at least one gaseous phase. The invention has applications, for example, in the fields of conversion and/or processing of distillates or residues produced by petroleum distillation, of liquid hydrocarbon batches produced by the liquefaction of carbon, of crude oils. More specifically, this equipment and this method make it possible to separate a mixture comprising a gaseous fraction and a liquid fraction possibly comprising solid particles, said mixture coming from a zone where a liquid batch, a gaseous batch, and solid particles come into contact. Although this equipment and this method may be applicable to any process requiring contact of a gaseous phase and a liquid phase with solid particles, the invention will be described below, in a nonlimiting way, for the particular case of hydroconversion of a batch of hydrocarbons in a boiling bed of solid catalytic particles.

The boiling bed method used for hydroconversion of heavy fractions of hydrocarbons or of a hydrocarbonic liquid batch made coming from a coal generally consists in placing in contact, most often by ascending co-current flow, a hydrocarbonic batch in liquid phase and a gaseous phase, in a reactor containing solid particles comprising a hydroconversion catalyst. The reaction zone generally includes at least one means of withdrawing the solid particles located near the base of the reactor and at least one means of supplying said particles containing a fresh catalyst near the top of said reactor. Said reactor further comprises most often at least one loop making it possible to recycle the liquid phase coming from the reaction zone, located inside or outside said reactor. Said recycling is intended, according to a method known to one skilled in the art, to maintain a level of expansion of the bed sufficient to guarantee proper functioning of the reaction zone in gas/liquid/solid triphasic operation.

According to a first principle, such as for example illustrated by patent application EP 1,086,734, the separation of the polyphasic mixture coming from the boiling bed is performed outside and downstream from a single reactor.

Patent application EP 732,389 proposes, according to another principle, the use of two reactors in series, including an external gas-liquid separator between said two reactors. The gaseous and liquid products coming from the first reactor are separated in said separator and a portion of said liquid products is recycled to said first reactor so as to maintain the boiling of the catalyst while the other part feeds the second reactor.

For economic reasons, the separator placed after the reactor is most often smaller than the reactor and the control of the liquid level in the external separator is consequently very difficult in this type of process. Further, the external separator must be at the same pressure as the reactor itself, implying an elevated fabrication cost of said separator.

### DETAILED DESCRIPTION OF THE INVENTION

An object of the invention is first of all equipment for processing hydrocarbons comprising at least two processing chambers and means of separating the liquid and gaseous fractions contained in the polyphasic mixture produced in the first processing chamber, in a zone (700) where a batch of liquid hydrocarbons, a gaseous batch, and solid particles come into contact, said equipment comprises:
a first processing chamber (1) comprising means (10) of introducing a batch of hydrocarbons and means (20) of introducing a gaseous batch into said zone (700), means inside said chamber (280, 310, 350) for separating gaseous and liquid fractions coming from said zone (700), means of collecting (210, 32) and means of removing (50) said liquid fraction toward the outside of said first chamber (1), means (70) of removing said gaseous fraction toward the outside of said first chamber (1) and means (130) of controlling the level of expansion of said liquid fraction inside said chamber (1),
means (50) of conveying at least a portion of said liquid fraction to a second reaction chamber (2),
a second processing chamber (2) comprising a second contact zone (701), and means of introducing a gaseous batch and means of introducing, into said second zone (701), a liquid batch comprising at least a portion of the liquid fraction produced in first processing chamber (1).

Said equipment can further comprise means for recycling a portion of said liquid fraction into the first chamber.

In one possible embodiment of the invention, the second processing chamber further comprises internal means for separating gaseous and liquid fractions produced in the second contact zone, means of collecting and means of removing said liquid fraction outside said second chamber, means of removing said gaseous fraction outside said second chamber and means of controlling the level of expansion of the liquid fraction in said second chamber. The device will thus advantageously further comprise means for recycling a portion of said liquid fraction in the second chamber.

The equipment described above advantageously further comprises means of controlling the level of expansion of the contact zones in said chambers.

The introducing means of the second zone can further comprise supply means for a liquid batch.

Said equipment preferably further includes means of controlling the liquid throughput conveyed between the two chambers, it can also further include means of controlling the gaseous throughput coming from said chambers.

According to one possible embodiment of the invention, at least the first chamber will, for example, comprise:
recycling equipment provided with at least one passage for the mixture comprising the gaseous fractions and liquid phase exiting the contact zone,
at least one separation element inside said chamber placed immediately after said passage(s) and making it possible to separate said mixture into a fraction A containing most of the liquid and a small portion of the gas and an essentially gaseous fraction B containing a small portion of the liquid,
a conduit placed near the upper part of said chamber and making it possible to remove fraction B,
a removal conduit that is an extension of the recycling equipment and through which fraction A flows by gravity and is removed,
means of controlling the level of expansion of said liquid phase in said chamber, said means being configured so as to maintain said level at a distance from the exit orifice of fraction B greater than or equal to 0.05 times the internal diameter of said chamber.

The invention also concerns a staged hydrocarbon processing method comprising separation of liquid and gaseous fractions contained in the polyphasic mixture produced by each stage of said process, said method comprising at least the following steps:
a) a hydrocarbon batch and a gaseous batch are introduced into a first processing chamber, and said hydrocarbon batch and said gaseous batch are placed in contact with solid particles,
b) inside said first reaction chamber, the liquid and gaseous fractions contained in the polyphasic mixture exiting step a) are separated,
c) the gaseous fraction and the liquid fraction are recovered separately outside the first chamber,
d) at least a portion of the liquid fraction coming from the first chamber is introduced into the second chamber with a gaseous batch and said liquid fraction and said gaseous batch are placed in contact, inside the second chamber, with solid particles.

Advantageously, the method further comprises the following steps:
e) inside said second chamber, the liquid and gaseous fractions contained in the polyphasic mixture exiting step d) are separated,
f) the gaseous fraction and the liquid fraction are recovered outside said second chamber.

In an embodiment of the method in which n chambers are used, n being between 2 and 10, at least a portion of the liquid fraction coming from a chamber n-1 placed upstream is introduced into chamber n placed downstream in the direction in which-the fluids are circulating.

According to the invention, said solid particles, when moving, will for example be able to be dispersed in the form of a suspension immersed in a liquid phase in a zone below the chamber, thus generally known by the Anglo-Saxon term reactor slurry. According to a preferred application of this invention, said solid particles, when moving, will be able to be present within a boiling bed. Examples of reactors that function according to the principles associated with slurry beds and boiling beds, as well as their main applications are described, for example, in “Chemical Reactors, P. Trambouze, H. Van Landeghem and J. P. Wauquier, ed., Technip (1988).” According to the invention, advantageously, in at least the first reaction chamber (i.e., the one comprising the internal separation), said solid particles are in a boiling or a slurry bed. Preferably the second (or other) chamber also operates as a boiling or slurry bed (respectively).

More particularly, but in a nonlimiting way, this invention has its application in the conversion of a batch introduced into said chamber in liquid form and containing hydrocarbons, said conversion being performed by reaction with a gaseous phase comprising, for example, hydrogen (hydroconversion) in the presence of a solid phase that most often provides catalytic activity (catalyst).

The batches that can be processed in the scope of this invention can be atmospheric residues or vacuum residues of direct distillation, deasphalted residues, residues derived from the conversion method such as, for example, those coming from coking, liquid batches of hydrocarbons produced by coal liquefaction, crude oils, from a fixed bed hydroconversion such as those produced by HYVAHL processing methods for bottoms in a boiling bed such as those produced by H-OIL methods, or even oils deasphalted by solvent, for example, propane, butane, or pentane, or even asphalts that usually come from deasphalting vacuum residues from direct distillation or vacuum residues from H-OIL or HYVAHL methods diluted by a hydrocarbonic fraction or a mixture of hydrocarbonic fractions selected from the group consisting of a light cycle oil (LCO according to the abbreviation of the Anglo-Saxon nomenclature light cycle oil),.a heavy cycle oil (HCO according to the abbreviation of the Anglo-Saxon nomenclature heavy cycle oil), a decanted oil (DO according to the abbreviation of the Anglo-Saxon nomenclature decanted oil), a slurry and gas oil fractions, notably those obtained by vacuum distillation, known by the Anglo-Saxon terminology VGO (vacuum gas oil). The batches can also be formed by mixing these various fractions in any proportions, notably of atmospheric residues and vacuum residues. They can also contain distillation cuts of gas oils and heavy gas oils produced by catalytic cracking having generally a distillation range of about 150° C. to about 370° C. or even 600° C. or more than 600° C. They can also contain aromatic extracts obtained during the production of lubricating oils. According to this invention, the batches that are processed are preferably atmospheric residues or vacuum residues, or mixtures of these residues.

The invention can also be applied, for example, to hydroprocessing methods for hydrocarbon batches such as the methods of hydrodesulfuration, hydrodenitriding, hydrodemetallization or hydrodearomatization.

In its most general form, one of the objects of this invention is equipment comprising a series of chambers in which a chemical reaction is most often produced, making it possible to achieve separation of various fractions produced by said reactions and more particularly the separation of liquid effluents and gaseous effluents produced by said reactions.

The invention makes it possible, for example, simultaneously to guarantee minimal entrainment of the liquid in the gas and to produce a liquid practically free of gas at the outlet of the device.

The application of this equipment or of this method further makes it possible to achieve substantial gains in terms of the capacity of the unit and in terms of the yield of reaction products produced in said unit.

This invention further makes it possible to control, in a simple and efficient way, the conveyance of said liquid among several successive reactors.

Further, this invention makes it possible to simplify the method and reduce the cost of the equipment since no supplementary separation means is necessary between the two reactors.

This invention will further be able to be applied easily and at a low cost in the field of modernizing already existing units.

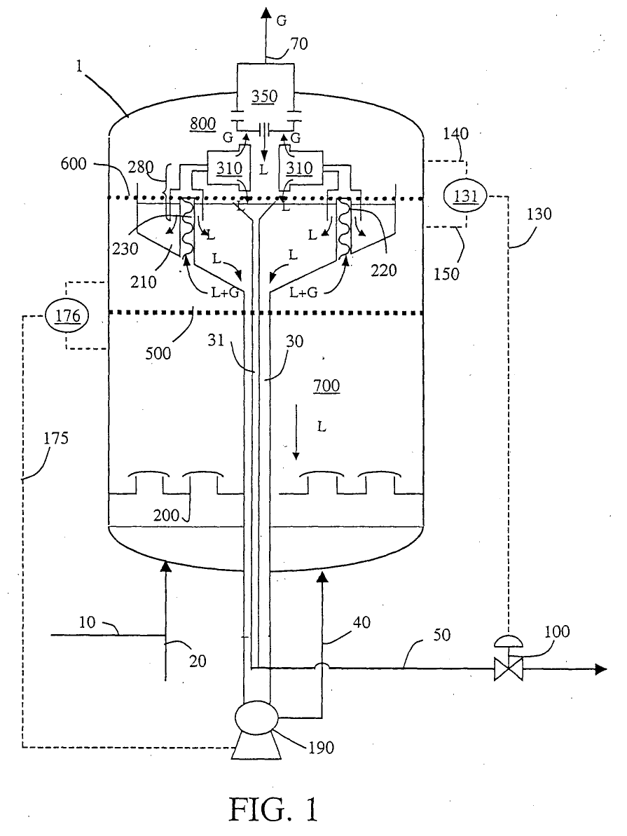

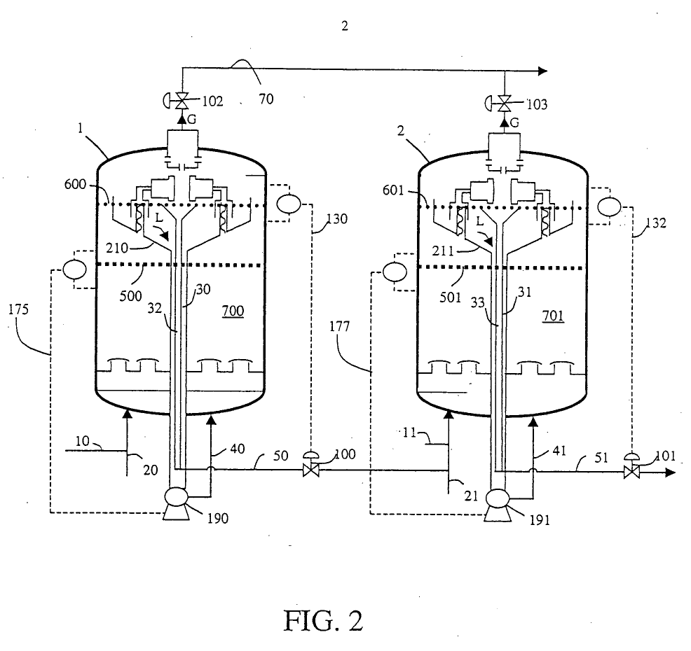

Other characteristics, details, and advantages of the invention will appear more clearly from reading the following description, made with the help of FIGS. 1 and 2, of a nonlimiting embodiment of the invention.

FIG. 1 diagrammatically illustrates an embodiment of a first chamber equipped with devices for internal separation of gaseous and liquid fractions at the outlet of a boiling bed in a first chamber.

FIG. 2 illustrates an embodiment of this invention in which two reaction chambers are used in series.

FIG. 1 illustrates, according to principles consistent with those already described in patent application FR 01/09055, a nonlimiting way of implementing a hydroconversion reactor 1 for a large batch of hydrocarbons in the presence of hydrogen (H₂) and catalytic particles within a boiling bed 700, able to be used to achieve this invention. At the top of reactor 1 an internal device for gas/liquid separation is installed.

For example, such a separation device for a mixture comprising at least one gaseous phase and one liquid phase can be integrated in a chamber and comprise at least:
equipment such as a recycling trough provided with at least one passage for said mixture, said passage being able to comprise, for example, a shaft preferably comprising inserts causing a centrifugal movement within said shaft(s),
a first separation element or primary separator placed immediately after said shafts(s) and making it possible to separate said mixture into a fraction A containing most of the liquid and a small amount of the gas and an essentially gaseous fraction B containing a small amount of the liquid,
a first conduit placed near the upper part of said chamber, making it possible to remove essentially gaseous fraction B coming out of the primary separator,
a second conduit that is a continuation of the recycling trough and through which fraction A containing most of the liquid coming out of said primary separator flows by gravity and is removed, said device further comprising means (130) to control the level of expansion of said liquid phase in said chamber, said means being configured so as to maintain the said level at a distance from the exit orifice of fraction B greater than or equal to 0.05 times the internal diameter of said chamber, preferably greater than 0.1 times said diameter. Said distance will, according to the invention, be as small as technically possible, and generally be between 0.05 and 20 times said diameter. Said second conduit (for removal of fraction A) can comprise separate means for recycling and withdrawing said fraction.

Of course this invention is not however limited to use of such a device and any known equivalent gas/liquid separation device will be able to be used without going beyond the scope of the invention.

Said reactor 1 is supplied with the liquid batch (comprising, for example, a mixture of heavy hydrocarbons) by a conduit 10 and with the gaseous batch (comprising, for example, mostly hydrogen) by a conduit 20, the mixture of the two phases being able to be performed either upstream from reactor 1 (according to FIG. 1) or in the reactor itself. The terms “upstream” and “downstream” are defined in this description in terms of the direction of the fluid circulation in this device. Equipment 200 used for the distribution of the liquid and gaseous batches is placed in the lower part of the reactor. After passage through equipment 200, the liquid and gaseous batches are placed in contact, within a boiling bed 700, with solid catalytic particles of known size and shape and optimized by one skilled in the art for implementing the reaction in question. The catalytic particles are kept in suspension by the upward speed of the liquid phase. Most often, said speed determines the rate of expansion of the bed of solid catalytic particles. Level 500, consisting of a slightly turbulent plane because of the agitation caused by the passage of gas and liquid bubbles, delimits the zone that is rich in solid catalytic particles. This level, for a given inventory of solid particles, most often depends essentially on the liquid throughputs and, to a lesser extent, on the gas throughputs passing through the reaction zone. Above level 500, said particles are not significantly entrained. The zone located between interface 500 delimiting the bed of said particles and an interface 600 consists overwhelmingly of a mixture of a gaseous fraction and a liquid fraction. The gaseous fraction generally flows in the form of bubbles in the continuous liquid fraction. Above interface 600, there is an essentially gaseous phase in a zone 800, containing only liquid projections dispersed in the continuous gaseous phase.

The catalyst is brought to boil by recycling the liquid fraction coming out of zone 700 in the reactor so as to maintain a surface speed of the liquid that is adapted to the kinetic constraints of the reaction. For this purpose, the liquid is withdrawn from the reactor below interface 600 thanks to collecting equipment 210 that can be a recycling trough provided with conduits 220 comprising inserts making it possible for fluids to pass and for the removal of most of the solid particles. Said equipment 210 is placed most often about in the center of reactor 1 and preferentially has in its upper part an approximately conical shape, for example such as described in U.S. Pat. No. 4,221,653. As represented in FIG. 1, said equipment 210 is immersed at least partly, and most often mostly, in the zone delimited by interfaces 500 and 600 and containing an essentially liquid phase. The liquid coming out of equipment 210 feeds a descending conduit 30, then a pump 190. When pump 190 lifts, the liquid is conveyed by a conduit 40 and reinjected into the reactor upstream from distribution means 200. According to a preferred embodiment of the invention and as diagramed in FIG. 1, a conduit 31 is installed inside conduit 30, and the upper part of conduit 31 is immersed in the essentially liquid phase, which makes it possible to recover the liquid located near liquid level 600 and to send it outside reaction chamber 700.

Collecting equipment 210 is connected to a series of devices, making it possible to promote gas-liquid separation of the products coming out of boiling bed 700, the entrainment of gas with the recycled liquid being damaging in a known way to the proper functioning of pump 190. To promote the coalescence of small bubbles and thus the separation of the gaseous fraction from the liquid fraction, advantageously one or several separation stages (280, 310, 350) are used inside reactor 1, consisting, for example, of cyclone-type equipment that promotes a vortex effect in the flow or any equivalent device. Separation stages (280, 310, 350) are located in the upper part of chamber 1, i.e., at least partially in zone 800 containing the essentially gaseous phase.

Collecting equipment 210, which makes it possible to feed conduit 30 and recycling system (190, 40) as described above, is also located inside reactor 1 and advantageously has an approximately axial symmetry, generally conical, around the axis of the chamber surrounding reactor 1 and covers at its largest dimension a cross section generally representing between 50 and 100% of the cross section of the reactor and preferably between 80 and 98% of this cross section. Pump 190 is preferably located outside the reactor but, without going beyond the scope of the invention, can also be inside said chamber. The nonrecycled part inside reactor 1 of liquid products from the reaction is withdrawn by conduit 50 which is an extension of conduit 31 as represented in FIG. 1. Of course this invention is not limited to this embodiment and it would be possible, for example, without going beyond the scope of the invention, to perform said withdrawal of liquid products directly in the recycling loop, either upstream from pump 190 (by connection into conduit 30) or downstream from it (by connection into conduit 40).

The gas coming out of the separation stages and relieved of most of the liquid is removed at the top of the chamber by a conduit 70 to be subjected to possible subsequent processing. Generally, the gas circulating in conduit 70 contains less than 10% by mass of liquid, preferably less than 7% and very preferably less than 1%, thanks to the combined use of separation means 280, 310, and 350.

The liquid products coming out of conduit 30 and essentially relieved of most of the gas are recycled downstream from the reactor via a recycling line 40 for liquid and under the effect of a recycling pump 190. The liquid effluent contains very small quantities of gas, generally less than 2% by mass and most often less than 1% by mass, thanks to separation stages 280, 310, and 350 used above recycling trough 210.

To control, according to a possible embodiment of the invention, the level of interface 500 inside reactor 1, measurement and control means are used to guarantee the proper functioning of the reactor. It can be envisioned, for example, that the level of interface 500 be measured precisely by evaluating the density profile in the reactor with the help of a gamma meter. The measurement of the level obtained using this means can be supplied to a regulation loop 175 which, by comparison with the setpoint that the operator inputs by a command device 176, controls the rotation speed of recycling pump 190. Thanks to such a system, a lowering of the level of interface 500 with respect to a given setpoint is corrected by increasing the rotation speed of pump 190, which induces an increase in the throughput of recycled liquid in the recycling loop and results in the increase of the surface speed of liquid in the reactor and consequently of the expansion rate of the catalyst bed.

To control the level of interface 600 of reactor 1, it can be envisioned, for example, that the density profile in the reaction is evaluated, typically with the help of a differential pressure sensor connected to the reactor by two pressure pickups, 140, located slightly above the desired interface and 150, located slightly below this same interface. To prevent clogging of these pressure pickups, known and controlled quantities of gas (for example, the initial gas batch) or liquid (for example, the recycled liquid) can be injected at the level of the pressure pickups. The measurement of the level obtained this way feeds a regulation loop 130 which causes, for example by the action of a command element 131, the opening of a slide valve 100 located on a removal conduit 50 for the liquid products of the reactor. As described above, said conduit 50 is preferably placed after conduit 31 located in conduit 30. With such a system, a lowering of interface 600 below a given setpoint will be corrected by at least a partial closing of slide valve 100, a decrease in the throughput of liquid withdrawn by line 50 and consequently an increase in the recycled liquid fraction and thus a raising of the position of interface 600 in reactor 1 if the gas and liquid feed throughputs in lines 10 and 20 remain stable. A raising of interface 600 is counterbalanced by pouring, into conduit 31, the liquid located above this interface. Slide valve 100 serves only to control the pressure difference between chamber 700 and the downstream equipment.

FIG. 2 illustrates an embodiment of the invention comprising equipment in which two hydroconversion reactors are arranged in a series. Shown in FIG. 2 are two reactors 1 and 2, the effluent liquid coming out of reactor 1 being used as the liquid batch for reactor 2.

According to the principles as defined above in relation to FIG. 1, the liquid batch is carried by conduit 10 and mixed into the hydrogen flow being conveyed by line 20 before being injected into reactor 1. Gas-liquid separation system 210 makes it possible to maintain liquid level 600 at a constant height and to recycle the liquid containing less than 1% by mass of gas to pump 190, making it possible for bed 700 to boil. At the outlet of separation device 210, the gas at the top of the reactor, essentially relieved of any liquid fraction, is sent by line 70 to the gas purification loop at the same time as the gaseous effluent coming out of reactor 2. Pump 190 guarantees recycling, by line 40, of a portion of the liquid products coming out-of the gas/liquid separation devices and collected by equipment 210, toward recycling conduit 30. Line 50, which is an extension of conduit 32 and is located upstream from pump 190, makes it possible to move the nonrecycled liquid fraction coming out of first reactor 1 toward reactor 2.

Without going beyond the scope of the invention, an extra batch of liquid hydrocarbons can be injected at the inlet to the second reactor by a line 11.

According to the invention, the liquid batch introduced into reactor 2 can consist partly or entirely of the liquid effluent coming out of reactor 1. Said reactor 2 is further fed with a gaseous batch (comprising mainly hydrogen) by a conduit 21, the mixture with the liquid phase(s) (containing the non-recycled liquid fraction and possibly an extra batch of liquid hydrocarbons) being able to be performed either upstream from reactor 2 according to FIG. 2 or in the reactor itself. The transport of the liquid from conduit 32 toward reactor 2 can be advantageously effected by pressure difference, the pressure of reactor 1 being at a slightly higher value than that of reactor 2. The pressure difference between both reactors 1 and 2 will be able to be regulated, for example, by opening or closing slide valves 102 and 103, placed on conduit 70. The throughput of the liquid phase coming out of reactor 1 can advantageously be regulated by slide valve 100 placed on conduit 50 without disturbing the flow. In reactor 2, the batch conveyed by conduit 50 is mixed with a gas stream 21. After reaction in a boiling bed 701, recycling elements 211 and one or more separation stages of the same type as those provided in reactor 1 guarantee the separation and removal of gaseous and liquid fractions derived from the reaction products. The liquid phase is conveyed by conduit 31 toward boiling pump 191 and the gaseous phase is removed at the top of the reactor to rejoin the gaseous effluent coming out of reactor 1. When pump 191 lifts, line 41 guarantees the recycling of the liquid in reactor 2. Part of the liquid coming out of reactor 2 via conduit 33 and line 51 is either collected or routed to a possible subsequent processing, for example to a third reactor identical in its structure and its function to those preceding. Slide valve 101 further makes it possible to maintain a constant liquid level 601 in reactor 2 by recycling a fraction matching the liquid fraction exiting said reactor 2. Devices for controlling levels 132 and 177, similar in their structure and functioning to devices 130 and 175 already described with respect to FIG. 1, make it possible to control the levels of interfaces 601 and 501 of reactor 2.

FIG. 2 illustrates the advantages derived from using separation devices according to the invention when several reactors are used in series. In implementing the conversion methods using boiling beds, it is advantageous to place two reactors in series to optimize the transformation of the liquid batch. The use of one or several internal separation stages (280, 310, 350), possibly combined with control of the level of interfaces 600, 601 respectively by means 130, 132 in reactors 1 and 2, makes it possible to withdraw all the gaseous products, notably the unconsumed hydrogen and the lightest hydrocarbons produced by the hydroconversion reaction, by conduit 70 and to send to reactor 2 only the liquid batch coming out of the first reactor via lines 50 without intermediate separation between the two reactors.

Thus, according to the invention illustrated by the embodiment of FIG. 2, for a constant volume of the second reactor, the separation according to the invention of the gaseous and liquid effluents inside the first chamber makes it possible to introduce, into the first reactor, a larger stream of the liquid batch to be processed and thus to greatly increase the capacity of the unit. Further, such an increase in capacity can, according to the invention, be achieved without the surface speed of the gasses within the reaction beds significantly entraining catalyst particles in the recycling loop.

Comparison of the following examples illustrates the advantages tied to this invention. Example 1 (comparative) gives the results obtained with a unit used in a conventional way in hydroconversion of residues.

Example 2 makes it possible to compare the conversions obtained with a unit functioning according to this invention.

Example 3 makes it possible to compare the capacities obtained with a unit functioning according to this invention.

These examples are the result of experiments performed in pilot units functioning as a boiling bed. Other experiments were performed in a slurry type reactor with a suitable pump and the same type of results were obtained.

### EXAMPLE 1

### Comparative

A high-vacuum residue [RSV in French—“residu sous vide”] of Safaniya origin is processed. The density is 15° C. and 1.048. All the yields are calculated starting with a base of 100 (by mass) of the RSV batch.

The high vacuum residue is processed in a pilot unit comprising two reaction chambers placed in series and operating according to the principles of boiling beds. All of the initial RSV batch is introduced into the first reactor, and all of the effluents coming out of the first reactor are reinjected directly into the second reactor without intermediate separation. Each reactor has a total volume of 2 liters.

This pilot unit simulates an industrial hydroprocessing unit for high vacuum residue in a boiling bed according to the H-Oil method marketed by the Axens company. The flow of the fluids is upward in this reactor as in the industrial unit.

The operating conditions are the following:
batch throughput (first reactor): 1.4 l/h at P=0, 1 MPa, T=15° C. (or 2 l/h under experimental conditions),
total pressure: 156 absolute bars (first and second reactor)
hydrogen stream: 560 l/h at P=0, 1 MPa and T=15° C. (or about 8.7 l/h under reaction conditions) at the inlet to the second reactor, 280 l/h at P=0, 1 MPa and T=15° C. (or about 4.3 l/h under reaction conditions),
temperature within the two reactors: 410° C.

The catalyst used in the two reactors is an NiMo catalyst.

The products coming out of the second reactor are fractionated in the laboratory into a gasoline fraction whose distillation range is between the initial point (IP) and 150° C., a gas oil fraction with a distillation range between 150 and 375° C., a vacuum distillate fraction (range is 375-540° C.) and a residual fraction (distillation point higher than 540° C.).

The various throughputs (in percent by mass) of the products obtained at the outlet of the second reactor after fractionation are:

| Product | Yields |
|---|---|
| Sulfurous hydrogen | 4.3 |
| Gas | 2.0 |
| Gasoline (IP-150° C.) | 2.5 |
| Diesel (150-375° C.) | 14.8 |
| Vacuum gas oil (375-540° C.) | 24.6 |
| Residue (greater than 540° C.) | 51.8 |

### EXAMPLE 2

### According to the Invention

The batch is the same as the one used in the preceding example. The operating conditions remain identical to those of example 1. Inside the first chamber, according to the invention, a separation device is used that makes it possible to separate the gaseous and liquid effluents so that the liquid fraction comprises less than 1% by weight of the gaseous fraction, the size of the reactors and the volume of the catalytic beds remain identical.

Thanks to the device, the volume of gaseous effluents introduced into the second reactor is considerably reduced compared to that of example 1 where all the hydrocarbon gasses produced in the first stage were reinjected into the second reactor. The only gaseous throughput at the inlet of the second reactor consists of the very small gas throughput coming from the first reactor (less than 1% by weight) and of the additional hydrogen throughput in the second reactor (280 l/h at P=0.1 MPa and T=15° C.) or about 4.3 l/h under reaction conditions. The throughput of the liquid batch entering the second reactor consists exclusively of the liquid fraction withdrawn from the first reactor.

The various throughputs of the products obtained at the outlet of the second reactor are, after fractionation, identical to that of example 1:

| Product | Yields |
|---|---|
| Sulfurous hydrogen | 4.1 |
| Gas | 2.0 |
| Gasoline (C5-150° C.) | 2.3 |
| Diesel (150-375° C.) | 18.1 |
| Vacuum gas oil (375-540° C.) | 32.7 |
| Residue (540° C.+) | 40.8 |

The comparison of examples 1 and 2 makes it possible to show that this invention makes it possible to reduce the final residue content by 11%.

This invention thus makes it possible significantly to increase the conversion of residue while in addition preserving an approximately constant fraction of the lightest hydrocarbons coming out of the hydroconversion method.

### EXAMPLE 3

### According to the Invention

This example makes it possible to show the improvement that is possible, by applying this invention, in the capacity of units for a constant reaction volume, for example in the scope of retrofitting (revamping according to the English term) an already existing unit.

To process a batch that is the same as in example 1, two reactors of the same dimensions as those of example 1 are used. The gaseous products are separated from the liquid inside the first reactor according to features identical to those of example 2. The liquid coming from the separation is injected into the second reactor with additional purified hydrogen corresponding to the amount necessary to obtain an overall conversion level of the unit identical to that of example 1.

Thus, at constant reactor volume, the volume of gaseous hydrocarbons separated during the first stage of the reaction in the first chamber makes it possible to inject a larger amount of the initial batch of (RSV) for a constant residue conversion rate at the outlet of the second reactor. In this case, for a final residue rate identical to 51.8% at the outlet of the, second chamber, the capacity of the unit of example 1, modified according to the invention, is increased significantly: a batch throughput of 3.7 l/h under experimental conditions (or a total batch throughput of 2.6 kg/h) is obtained, or an increase of 1.2 kg/h compared to the conditions of example 1.

The application of this equipment thus makes it possible to increase the capacity of the unit by about 85%.

1. Equipment for processing hydrocarbons comprising at least two processing chambers and means of separating the liquid and gaseous fractions contained in the polyphasic mixture produced in the first processing chamber, in a zone (700) where a batch of liquid hydrocarbons, a gaseous batch, and solid particles come into contact, said equipment being characterized in that it comprises:
a first processing chamber (1) comprising means (10) of introducing a batch of hydrocarbons and means (20) of introducing a gaseous batch into said zone (700), means inside said chamber (280, 310, 350) for separating gaseous and liquid fractions coming from said zone (700), means of collecting (210, 32) and means of removing (50) said liquid fraction toward the outside of said first chamber (1), means (70) of removing said gaseous fraction toward the outside of said first chamber (1) and means (130) of controlling the level of expansion of said liquid fraction inside said chamber (1),—means (50) of conveying at least a portion of said liquid fraction to a second reaction chamber (2),
a second processing chamber (2) comprising a second contact zone (701), and means of introducing a gaseous batch and means of introducing, into said second zone (701), a liquid batch comprising at least a portion of the liquid fraction produced in first processing chamber (1).

2. Equipment according to claim 1, further comprising recycling means (30, 190, 40) in first chamber (1) for a portion of said liquid fraction.

3. Equipment according to claim 1, in which said second chamber further comprises internal means for separating the gaseous and liquid fractions coming from second contact zone (701), collecting means (211, 33) and means (51) for removing said liquid fraction toward the outside of said second chamber (2), means (70) for removing said gaseous fraction toward the outside of said second chamber (2) and means (132) for controlling the level of expansion of the liquid fraction in said second chamber (2).

4. Equipment according to claim 3, further comprising means (31, 191, 41) for recycling, in second chamber (2), a portion of said liquid fraction.

5. Equipment according to claim 1, further comprising means (175, 177) for controlling the levels of expansion of contact zones (700, 701) in said chambers (1, 2).

6. Equipment according to claim 1, in which said introduction means of the second chamber further comprise additional means (11) for the liquid batch.

7. Equipment according to claim 1, further comprising means (100) for controlling the throughput of the liquid transferred between both chambers (1, 2).

8. Equipment according to claim 1, further comprising means (102, 103) for controlling the gaseous throughput coming from said chambers (1, 2).

9. Equipment according to claim 1, in which at least the first chamber comprises:
recycling equipment (210) provided with at least one passage for said polyphasic mixture,
at least one separation element inside said chamber (280) placed immediately after said passage(s) and making it possible to separate said mixture into a fraction A containing most of the liquid and a small portion of the gas and an essentially gaseous fraction B containing a small portion of the liquid,
a conduit (70) placed near the upper part of said chamber and making it possible to remove fraction B,
removal conduit (30) that is an extension of the recycling equipment (210) and through which fraction A flows by gravity and is removed,—means (130) of controlling the level of expansion of said liquid
phase in said chamber, said means being configured so as to maintain said level at a distance from the exit orifice of fraction B greater than or equal to 0.05 times the internal diameter of said chamber.

10. Staged processing method for hydrocarbons, comprising a separation of liquid and gaseous fractions contained in the polyphasic mixture produced at each stage of said process, said method being characterized in that it comprises at least the following stages:
a) a hydrocarbon batch and a gaseous batch are introduced into a first processing chamber, and said hydrocarbon batch and said gaseous batch are placed in contact with solid particles,
b) inside said first reaction chamber, the liquid and gaseous fractions contained in the polyphasic mixture exiting step a) are separated, c) the gaseous fraction and the liquid fraction are recovered separately outside the first chamber,
d) at least a portion of the liquid fraction coming from the first chamber is introduced into a second chamber with a gaseous batch and said liquid fraction and said gaseous batch are placed in contact, inside the second chamber, with solid particles.

11. Method according to claim 10 in which:
e) inside said second chamber, the liquid and gaseous fractions contained in the polyphasic mixture exiting step d) are separated,
f) the gaseous fraction and the liquid fraction are recovered outside said second chamber.

12. Method according to claim 10 in which n chambers are used, n being between 2 and 10, at least a portion of the liquid fraction coming from a chamber n-1 placed upstream being introduced into chamber n placed downstream in the direction in which the fluids are circulating.

13. Method according to claim 10 for hydroprocessing or hydroconversion of a batch or a mixture of batches included in the group consisting of atmospheric residues or vacuum residues from direct distillation, deasphalted residues, residues derived from the conversion method, hydroprocessing methods for bottoms with boiling bed, oils deasphalted by a solvent, asphalts, a mixture of hydrocarbonic fractions selected from the group formed by a light cycle oil, a heavy cycle oil, a decanted oil, a slurry and gas oil fractions, notably those obtained by vacuum distillation.

14. Method according to claim 10, operating with a boiling bed, at least in the first reaction chamber.

15. Method according to claim 10, operating with a slurry bed in at least the first reaction chamber.
